(19) United States
(12) Patent Application Publication
(10) Pub. No.: US 2010/0094038 A1
(43) Pub. Date: Apr. 15, 2010

(54) SYNTHESIS

(75) Inventors: Antony Warr, Wirral Merseyside (GB); Peter McCormack, Wirral Merseyside (GB); Jonathan Eddolls, Wirral Merseyside (GB); Elliot Latham, Wirral Merseyside (GB)

Correspondence Address: KNOBBE MARTENS OLSON & BEAR LLP, 2040 MAIN STREET, FOURTEENTH FLOOR, IRVINE, CA, 92614, US

(73) Assignee: PHOENIX CHEMICALS LIMITED, WIRRAL MERSEYSIDE (GB)

(21) Appl. No.: 12/522,899

(22) PCT Filed: Jan. 8, 2008
(86) PCT No.: PCT/EP2008/050152

§ 371 Date: Dec. 11, 2009

(30) Foreign Application Priority Data
Jan. 11, 2007 (GB) 0700515.0

(51) Int. Cl. C07C 303/24

(52) U.S. Cl. 558/46

(57) ABSTRACT

The present invention provides a method of preparing a sulfonyl ether comprising the steps of: v) reacting a sulfonic acid with an anhydride, under continuous vacuum distillation conditions, to form a carboxysulfonate; vi) reacting the carboxysulfonate with an optionally substituted cycloalkane ring containing at least 3 heteroatoms to form a sulfonyl ether pre-mix comprising a sulfonyl ether and at least two additional reaction products each containing at least one ether linkage; vii) subjecting the pre-mix to continuous vacuum distillation conditions to remove at least some of the additional reaction products from the sulfonyl ether pre-mix to provide a crude sulfonyl ether product; and viii) purifying the crude sulfonyl ether product.

This invention relates to a process for synthesising sulfonyl ethers. More particularly, this invention relates to a process of synthesizing sulfonyl ethers from sulfonic acids.

Sulfonyl ethers have numerous applications and are employed in the synthesis of compounds for use in a wide range of technical fields. For example, as confirmed in Japanese Patent Nos. 03-178958, 03-178959, 03-123768 and 03-123769, sulfonyl ethers may be used in the synthesis of methyl ether derivatives which are useful in the field of photography as gelatin membrane hardeners, development accelerators and chemical sensitizers. The use of sulfonyl ethers as photographic gelatin hardeners is also disclosed in U.S. Pat. No. 4,100,200.

Additionally, as disclosed in UK Patent Application No. 0616865.2, sulfonyl ethers may be used in the synthesis of HI 6, a bis-pyridinium oxime antidote to certain organophosphate nerve agents.

As a result of the utility of sulfonyl ethers in the synthesis of a wide range of compounds, numerous synthetic pathways for sulfonyl ethers have been proposed. For example, in U.S. Pat. No. 4,100,200 and in Burness, Wright and Perkins, J. Org Chem 1977, 42, 2910, a synthesis of bis(methylsulfonoxymethyl) ether is disclosed in which acetyl methylsulfonate is reacted with trioxane to form bis(methylsulfonoxymethyl) ether which was then purified.

Further examples of syntheses of sulfonyl ethers are provided in Japanese Patent Nos. 03-178958, 03-178959, 03-123768 and 03-123769. In each of those patents, pathways for producing sulfonyl ethers from acetoxymethanesulfonate are disclosed.

While the synthetic pathways disclosed in the above-mentioned documents result in sulfonyl ethers of acceptable purity for use in the field of photography, in other fields, the purity requirement will be higher. It would therefore be desirable to provide a synthesis of high purity sulfonyl ethers.

Thus, according to a first aspect of the present invention, there is provided a method of preparing a sulfonyl ether comprising the steps of:
i) reacting a sulfonic acid with an anhydride, under continuous vacuum distillation conditions, to form a carboxysulfonate;
ii) reacting the carboxysulfonate with an optionally substituted cycloalkane ring containing at least 3 heteroatoms to form a sulfonyl ether pre-mix comprising a sulfonyl ether and at least two additional reaction products each containing at least one ether linkage;
iii) subjecting the pre-mix to continuous vacuum distillation conditions to remove at least some of the additional reaction products from the sulfonyl ether pre-mix to provide a crude sulfonyl ether product; and
iv) purifying the crude sulfonyl ether product.

Using this process, sulfonyl ether products having a purity of greater than 97% have been obtained. It will be understood by those skilled in the art that such high purity products may be used in the synthesis of a wider range of compounds than the sulfonyl ethers prepared according to the prior art methods discussed above.

In preferred embodiments, the sulfonic acid has the formula:

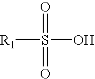

wherein R₁ is selected from the group consisting of hydrogen, substituted and unsubstituted branched and straight-chain alkyl, alkoxy, haloalkyl, alkoxylated alkyl, alkoxylated alkoxy, alkylamino, substituted and unsubstituted cycloalkyl, substituted and unsubstituted cycloalkylamino, substituted and unsubstituted carbocyclic aryl, substituted and unsubstituted carbocylic aryloxy, substituted and unsubstituted heteroaryl, substituted and unsubstituted carbocylic arylamino and substituted and unsubstituted heteroarylamino, wherein the or each heteroatom is independently selected from sulphur, nitrogen and oxygen. In a most preferred embodiment, R₁ is alkyl, more preferably methyl

In a preferred embodiment the anhydride has the formula:

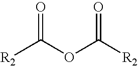

wherein R₂ are the same or different and are selected from the group consisting of hydrogen, substituted and unsubstituted branched and straight-chain alkyl, alkoxy, haloalkyl, alkoxylated alkyl, alkoxylated alkoxy, alkylamino, substituted and unsubstituted cycloalkyl, substituted and unsubstituted cycloalkylamino, substituted and unsubstituted carbocyclic aryl, substituted and unsubstituted carbocylic aryloxy, substituted and unsubstituted heteroaryl, substituted and unsubstituted carbocylic arylamino and substituted and unsubstituted heteroarylamino, wherein the or each heteroatom is independently selected from sulphur, nitrogen and oxygen. In the most preferred embodiment, R₂ is alky, more preferably methyl.

Preferably, the carboxysulfonate has the formula:

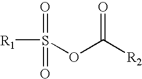

wherein R₁ and R₂ are as identified above.

The optionally substituted cycloalkane ring may take any form provided that it does not adversely affect the purity of the final sulfonyl ether. The cycloalkane ring is preferably 4 to 10 membered. The cycloalkane ring may be substituted with any substituent selected from the group consisting of hydrogen, substituted and unsubstituted branched and straight-chain alkyl, alkoxy, haloalkyl, alkoxylated alkyl, alkoxylated alkoxy, alkylamino, substituted and unsubstituted cycloalkyl, substituted and unsubstituted cycloalkylamino, substituted and unsubstituted carbocyclic aryl, substituted and unsubstituted carbocylic aryloxy, substituted and unsubstituted heteroaryl, substituted and unsubstituted carbocylic arylamino and substituted and unsubstituted heteroarylamino, wherein the or each heteroatom is independently selected from sulphur, nitrogen and oxygen. However, it is especially preferred that the cycloalkane ring is unsubstituted. For example, the cycloalkane ring may be trioxane or tetraoxane. In a most preferred embodiment, the cycloalkane ring is trioxane.

In a preferred embodiment, the sulfonyl ether has the formula:

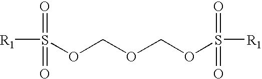

wherein R₁ is as identified above.

Most preferably, the sulfonyl ether is bis(methanesulfonylmethyl)ether.

The pre-mix includes at least two reaction products besides the sulfonyl ether, which each contain at least one ether linkage. The most likely additional reaction products which will be included in the pre-mix will have the formula:

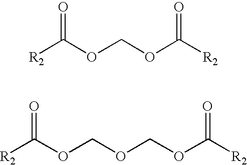

wherein R₂ is as identified above.

These additional products are removed using continuous vacuum distillation conditions and may be discarded or utilized in other syntheses.

The continuous vacuum distillation conditions used in steps i) and/or iii) may be provided using any apparatus known in the art which does not adversely affect the purity of the final sulfonyl ether product. However, in preferred embodiments, the continuous vacuum distillation conditions are provided using wiped film evaporator apparatus.

The crude sulfonyl ether product may be purified in any way. Preferably, a solvent/anti-solvent combination is employed. For example, the solvent/anti-solvent combination may be applied to the crude product to separate the sulfonyl ether product from impurities. The sulfonyl ether product could then be subjected to low temperature conditions to yield sulfonyl ether crystals which could then be filtered off, leaving the solvent and any other impurities. Additionally, the sulfonyl ether crystals could be washed to remove any adhered solvent/impurity.

In a particularly preferred embodiment, the crude sulfonyl ether product is charged to 1,2-dimethoxyethane (1.1 wt) at ambient temperature. To this a solvent/anti-solvent combination, for example a mixture of diisopropyl ether (10.0 wt) & tetrahydrofuran (0.06 wt), may be charged while maintaining the temperature at 10 to 15° C. Seed crystals may be added and crystallization occurs in this temperature range. The resulting slurry is cooled to 0-5° C. and stirred, preferably for at least an hour. It is then filtered and rinsed with, for example, DIPE/THF mixture (2×1.0 wt). The slurry is then filtered again, in a pressure (PALL) filter and is not exposed to air/moisture. The resulting cake is dried under a nitrogen stream.

The invention will now be more particularly described with reference to the following example.

### EXAMPLE 1

Synthesis of bis(methanesulfonylmethyl)ether from the reaction of acetoxymethanesulfonate with trioxane utilising continuous vacuum distillation.

Step 1

Synthesis of Acetoxymethanesulfonate (AMS)

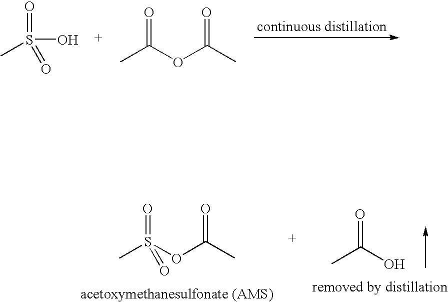

To acetic anhydride (AA) (1751 g, 17.1 mols, 3 mol equiv.) in a flask is added methanesulfonic acid (MSA) (550 g, 5.7 mols, 1 mol equiv.) with stirring. The resulting yellow solution is stirred for 20-30 min. The material is then pumped into an apparatus suitable for continuous vacuum distillation (wiped film evaporator 2 inch [5 cm] column diameter, flow rate; 9.9 ml/min). After appropriate priming of the apparatus distillate (1546 g) and residue (713 g) are collected. The residue is analysed by ¹H nmr (w/w %): 93.6 AMS, 4.0 MSA, 1.4 AA, 0.6 AcOH.

Step 2

Reaction of crude AMS with trioxane

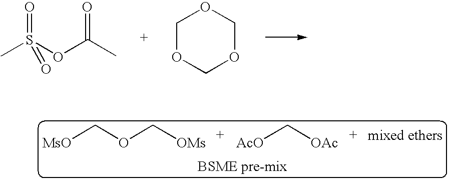

To crude AMS (710 g, 4.83 mol AMS) with good stirring was added solid trioxane (222.3 g, 2.47 mol, 0.5 mol equiv relative to AMS+AA) at such a rate to maintain a temperature of 60-65° C. At the end of the addition the reaction mixture was stirred for approximately 2 h at 60° C. Analysis of this material by ¹H nmr revealed it to be a complex mixture of mixed sulfonyl and acetyl ethers, methylene diacetate and product bis(methanesulfonylmethyl)ether BSME.

Step 3

Continuous vacuum distillation of the BSME pre-mix from step 2

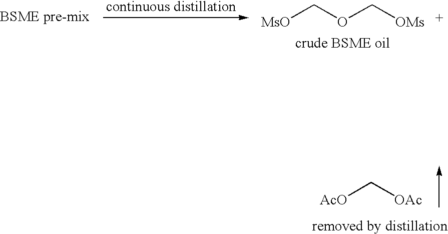

The crude BSME pre-mix liquid (932.6 g) is pumped into an apparatus suitable for continuous vacuum distillation (wiped film evaporator 2 inch [5 cm] column diameter, flow rate; 9.9 ml/min). After appropriate priming of the apparatus distillate is collected (270 g) and residue (573 g). The residue is analysed by ¹H nmr (w/w %): 62.6 BSME.

Step 4

Crystallization/purification of BSME

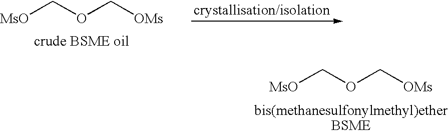

Crude BSME (345 g, 62.6% w/w) is charged to 1,2-dimethoxyethane (385 g) at ambient temperature. To this a mixture of diisopropyl ether (361 g) and tetrahydrofuran (19 g) are carefully charged whilst maintaining the temperature at 10-15° C. Some BSME seed crystals are added and crystallisation occurs in this temp range. The slurry is cooled to 0-5° C. and stirred for another hour, filtered and rinsed with DIPE/THF mixture (2×340 g). The slurry is filtered in a pressure (PALL) filter and is not exposed to air/moisture. The pale brown cake (165 g) is dried under nitrogen stream ready for the next step of the process. ¹H nmr CDCl₃: 5.53 CH2O (4H), 3.13 OS(O)₂CH₃ (6H).

1. A method for preparing a sulfonyl ether comprising:
i) reacting a sulfonic acid with an anhydride, under continuous vacuum distillation conditions, to form a carboxysulfonate;
ii) reacting the carboxysulfonate with an unsubstituted or substituted cycloalkane ring containing at least 3 oxygen heteroatoms to form a sulfonyl ether pre-mix comprising a sulfonyl ether and at least two additional reaction products each containing at least one ether linkage;
iii) subjecting the pre-mix to continuous vacuum distillation conditions to remove at least some of the additional reaction products from the sulfonyl ether pre-mix to provide a crude sulfonyl ether product; and
iv) purifying the crude sulfonyl ether product.

2. The method of claim 1 wherein the sulfonic acid has a formula: 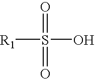
wherein R₁ is selected from the group consisting of substituted branched chain alkyl, unsubstituted branched chain alkyl, substituted straight-chain alkyl, unsubstituted straight-chain alkyl, alkoxy, haloalkyl, alkoxylated alkyl, alkoxylated alkoxy, substituted cycloalkyl, unsubstituted cycloalkyl, substituted carbocyclic aryl, unsubstituted carbocyclic aryl, substituted carbocylic aryloxy, and unsubstituted carbocyclic aryloxy.

3. The method of claim 2 wherein R₁ is methyl.

4. The method of claim 1, wherein the anhydride has a formula: 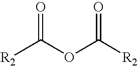
wherein R₂ are the same or different and are selected from the group consisting of hydrogen, substituted branched chain alkyl, unsubstituted branched chain alkyl, substituted straight-chain alkyl, unsubstituted straight-chain alkyl, alkoxy, haloalkyl, alkoxylated alkyl, alkoxylated alkoxy, substituted cycloalkyl, unsubstituted cycloalkyl, substituted carbocyclic aryl, unsubstituted carbocyclic aryl, substituted carbocylic aryloxy, and unsubstituted carbocyclic aryloxy.

5. The method of claim 3 wherein R₂ is methyl.

6. The method of claim 1, wherein the carboxysulfonate has a formula: 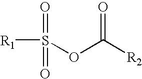
wherein R₁ is selected from the group consisting of substituted branched chain alkyl, unsubstituted branched chain alkyl, substituted straight-chain alkyl, unsubstituted straight-chain alkyl, alkoxy, haloalkyl, alkoxylated alkyl, alkoxylated alkoxy, substituted cycloalkyl, unsubstituted cycloalkyl, substituted carbocyclic aryl, unsubstituted carbocyclic aryl, substituted carbocylic aryloxy, and unsubstituted carbocyclic aryloxy; and R₂ is selected from the group consisting of hydrogen, substituted branched chain alkyl, unsubstituted branched chain alkyl, substituted straight-chain alkyl, unsubstituted straight-chain alkyl, alkoxy, haloalkyl, alkoxylated alkyl, alkoxylated alkoxy, substituted cycloalkyl, unsubstituted cycloalkyl, substituted carbocyclic aryl, unsubstituted carbocyclic aryl, substituted carbocylic aryloxy, and unsubstituted carbocyclic aryloxy.

7. The method of claim 6 wherein the carboxysulfonate is acetyl methane sulfonate

8. The method of claim 1, wherein the cycloalkane ring is trioxane.

9. The method of claim 1, wherein the sulfonyl ether has the formula: 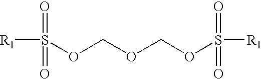
wherein R₁ is selected from the group consisting of substituted branched chain alkyl, unsubstituted branched chain alkyl, substituted straight-chain alkyl, unsubstituted straight-chain alkyl, alkoxy, haloalkyl, alkoxylated alkyl, alkoxylated alkoxy, substituted cycloalkyl, unsubstituted cycloalkyl, substituted carbocyclic aryl, unsubstituted carbocyclic aryl, substituted carbocylic aryloxy, and unsubstituted carbocyclic aryloxy.

10. The method of claim 1, wherein the sulfonyl ether is bis(methanesulfonylmethyl)ether.

11. The method of claim 1, wherein the continuous vacuum distillation conditions used in steps i) and/or iii) are provided using wiped film evaporator apparatus.

12. The method of claim 1, wherein the crude sulfonyl ether product is purified in step iv) using a solvent/anti-solvent combination.
